(19) United States
(12) Patent Application Publication
(10) Pub. No.: US 2021/0294908 A1
(43) Pub. Date: Sep. 23, 2021

(54) SHARING SYSTEM OF VETERINARY INFORMATION FOR PETS USING IDENTIFICATION CODE

(71) Applicant: Dr. Tail Inc., Seoul (KR)

(72) Inventor: Dae Hwa LEE, Suwon-si (KR)

(21) Appl. No.: 17/063,628

(22) Filed: Oct. 5, 2020

(30) Foreign Application Priority Data
Mar. 17, 2020 (KR) 10-2020-0032654

(51) Int. Cl. G06F 21/60; A61D 99/00; G06F 21/62; H04L 9/06; A01K 29/00

(52) U.S. Cl. CPC G06F 21/604; A61D 99/00; A01K 29/00; G06F 21/602; H04L 9/0643; G06F 21/6245

(57) ABSTRACT

The present disclosure relates to a sharing system of veterinary information for pets, which enables veterinary information for pets to be efficiently shared and used for the purpose of study or the like while protecting personal information of an owner through the use of an identification code. The sharing system registers the veterinary information for pets transmitted from a veterinarian terminal, and provides the registered veterinary information for pets to a study institution terminal, comprises: a veterinary information receiving part configured to receive the veterinary information for pets transmitted from the veterinarian terminal; a veterinary information delivering part configured to deliver the veterinary information for pets received at the veterinary information receiving part to an owner terminal; an owner code generating part configured to generate an owner code of an owner from owner's information included in the veterinary information for pets; a pet code generating part configured to generate a pet code from animal information included in the veterinary information for pets; a veterinary information registering part configured to register the veterinary information for pets, the owner code, and the pet code in a database server; and a veterinary information sharing part configured to process, in response to a request from the study institution terminal, owner's information in a de-identification manner and provide the veterinary information for pets registered in the database server to the study institution terminal.

### CROSS-REFERENCE TO RELATED APPLICATION

This application claims priority to and the benefit of Korean Patent Application No. 10-2020-0032654 filed in the Korean Intellectual Property Office on Mar. 17, 2020, the entire contents of which are incorporated herein by reference.

### BACKGROUND OF THE DISCLOSURE

### (a) Field of the Disclosure

The present disclosure relates to a sharing system of veterinary information for pets, which enables veterinary information of a pet to be efficiently shared and used for the purpose of study or the like while protecting personal information of an owner through the use of an identification code.

### (b) Description of the Related Art

A pet may be defined as an animal which lives with the human.

The Ministry of Agriculture, Food, and Rural Affairs of the Republic of Korea revealed that the portion of domestic households that raise pets was increased from 17% in 2012 to 28.1% in 2017. It is expected that, because of social factors, such as the heavy graying of the population structure, the growth of single households, a stressed society and the like, change in attitude as a family member, companion and partner for pets, and the like, the portion of households that raise the pets increases exponentially. With the growth of the pet raising households, the industry related to the pets, such as animal clinics, medical devices, various insurance products, and so forth, is also growing rapidly.

However, in spite of the growth of the pet raising households and the related industry, a registration system of the pets and a veterinary information sharing system of the pets are not yet established. For this reason, there are still problems in that the wrong behaviors for the pets, such as the abuse and neglect of the pets, the desertion and loss of the pets and the like, are not controlled in an efficient manner.

In addition, most animal clinics store veterinary information of the pets in the database provided in the respective clinics. For this reason, the owner who wants to view respective veterinary information needs to visit the respective clinics. Only when the owner requests to view respective veterinary information, the owner can confirm and utilize veterinary information on a limited basis.

Accordingly, in a case in which an animal clinic was going out of business, veterinary information stored in the animal clinic may be discarded. In addition, even in a case in which the owner moved to another place, or the pet was abandoned and was adopted by other owners, other owners or animal clinics can't refer to previous veterinary information. This may take additional economic costs and time for vaccination or disease treatment. Further, there is a problem that it is almost possible to find the owner of the abandoned (or lost) pet.

In addition, veterinary information for pets may be utilized as an important material for caring or studying other pets as well as the respective pet. However, veterinary information obtained in a specific animal clinic has not been shared with other animal clinics or institutions, which makes it impossible to utilize veterinary information in an efficient manner.

In general, an animal clinic stores and manages veterinary information of pets, including both veterinary information of a pet and information of an owner of a pet. In this case, when veterinary information of pets is shared, owner's information may also be shared. This may cause a problem that personal information of the owner leaks. Such leakage of personal information becomes the biggest hindrance to sharing veterinary information of pets.

Thus, in order to meet the growth of the pet raising households, there is a demand for a system which is capable of more systemically managing veterinary information for pets in a more efficient manner, and sharing veterinary information for pets to efficiently utilize veterinary information for pets while protecting personal information of an owner. Furthermore, from the viewpoint of public benefits in that experiments aimed directly for an animal, such as a biometric experiment, an activity experiment, and the like, is reduced, the sharing of veterinary information for pets is further required.

### SUMMARY

### Problem to be Solved

The present disclosure is proposed to solve the aforementioned problems, and the object of the present disclosure is to provide a sharing system of veterinary information for pets for sharing veterinary information for pets to efficiently utilize veterinary information for pets while protecting personal information of an owner.

Furthermore, the present disclosure provides a sharing system of veterinary information for pets, which enables veterinary information for pets to be stored, registered, and utilized according to the permission of an owner.

Furthermore, the present disclosure to provide a sharing system of veterinary information for pets enables veterinary information for pets to be more easily stored, managed and utilized.

In addition, the present disclosure provides a sharing system of veterinary information for pets, which is capable of further enhancing the security and integrity of veterinary information for pets that is to be stored and managed on a network.

### Solution to Problem

According to an embodiment for achieving the aforementioned object, there is provided a sharing system of veterinary information for pets for registering veterinary information for pets transmitted from a veterinarian terminal, and providing registered veterinary information for pets to a study institution terminal, comprising: a veterinary information receiving part configured to receive veterinary information for pets transmitted from the veterinarian terminal; a veterinary information delivering part configured to deliver veterinary information for pets received at the veterinary information receiving part to an owner terminal; an owner code generating part configured to generate an owner code of an owner from owner's information included in veterinary information for pets; a pet code generating part configured to generate a pet code from animal's information included in veterinary information for pets; a veterinary information registering part configured to register veterinary information for pets, the owner code, and the pet code in a database server; and a veterinary information sharing part configured to process, in response to a request from the study institution terminal, owner's information in a de-identification manner and provide veterinary information for pets registered in the database server to the study institution terminal.

Furthermore, in the veterinary information sharing system of the present embodiment, veterinary information for pets is configured to be identified by a composite identification code in which the owner code and the pet code are combined with each other.

Furthermore, in the veterinary information sharing system of the present embodiment, the pet code is generated using a species-based serial number of the pet.

Furthermore, in the veterinary information sharing system of the present embodiment, the veterinary information delivering part is configured to further transmit, to the owner terminal of the owner, registration permission request information for confirming whether the owner wants to register or sharing of veterinary information for pets or not and in response to a permission signal that requests the registration or sharing of veterinary information for pets, which is transmitted from the owner terminal of the owner, the veterinary information registering part is configured to register veterinary information for pets, and the veterinary information sharing part is configured to allow veterinary information for pets to be shared.

Furthermore, in the veterinary information sharing system of the present embodiment, the veterinary information delivering part includes: a veterinary information processing part configured to process veterinary information for pets in a form that is easy to be checked by the owner; an explanation information extracting part configured to extract explanation information for explaining veterinary information for pets, and a statistical information calculating part configured to calculate statistical information about a treatment item of veterinary information for pets.

Furthermore, in the veterinary information sharing system of the present embodiment, the veterinary information registering part includes: a de-identification processing part configured to process owner's information included in veterinary information for pets in a de-identification manner; a veterinary information encrypting/registering part configured to perform a hashing process and an encrypting process on veterinary information for pets and register processed information in a species-based database; and a hash value dispersedly-storing part configured to match a hash value, which is generated by processing the hashing process on veterinary information for pets at the veterinary information encrypting/registering part, with at least one of the owner code, the pet code, and the composite identification code, and dispersedly store matched information in a hash database.

### Effects of the Present Disclosure

According to the present disclosure in some embodiments, by using an owner code, a pet code, or a composite identification code in which the owner code and the pet code are combined with each other, it is possible to identify veterinary information for pets and process information of an owner in a de-identification manner, thus sharing and efficiently utilizing veterinary information for pets while protecting personal information of an owner.

Furthermore, according to the present disclosure, it is possible to store, register and manage veterinary information for pets in accordance with the permission of an owner and to prevent veterinary information for pets from leaking and being used without permission.

In addition, according to the present disclosure, it is possible to automatically store, register, and provide veterinary information for pets in a system, thus more easily managing and utilizing veterinary information of pets.

In addition, according to the present disclosure, it is possible to manage and dispersedly store veterinary information for pets while performing a hashing process and an encrypting process on veterinary information of pets, thus enhancing security and integrity of veterinary information for pets to be stored and managed on a network.

### BRIEF DESCRIPTION OF THE DRAWINGS

FIG. 1 is a block diagram showing a network configuration of a sharing system of veterinary information for pets according to an embodiment of the present disclosure.

FIG. 2 is a block diagram showing a major configuration of the veterinary information sharing system according to an embodiment of the present disclosure.

FIG. 3 is a view showing an example of veterinary information provided with owner's information processed in a de-identification manner, according to an embodiment of the present disclosure.

FIG. 4 is a control flowchart illustrating a process of sharing veterinary information for pets according to an embodiment of the present disclosure.

### DETAILED DESCRIPTION

Specific structural or functional descriptions of embodiments according to the inventive concepts disclosed herein are merely illustrated for the purpose of describing embodiments according to the concepts of the present disclosure. The embodiments, according to the concepts of the present disclosure may be practiced in various forms and are not limited to embodiments described herein.

In addition, embodiments according to the concepts of the present disclosure may be embodied with various modifications and may have various forms. Thus, the embodiments will be described in detail with reference to the accompanying drawings. However, the embodiments according to the concepts of the present disclosure are not intended to limit specific embodiments and may include all modifications, equivalents, or alternatives falling within the spirit and scope of the present disclosure.

Preferable embodiments of the present disclosure will now be described in detail with reference to the accompanying drawings.

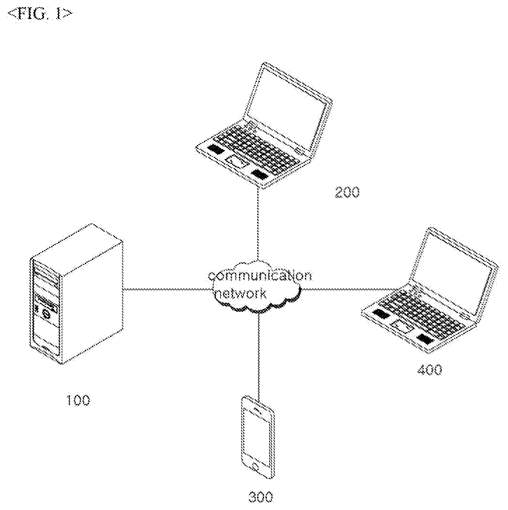

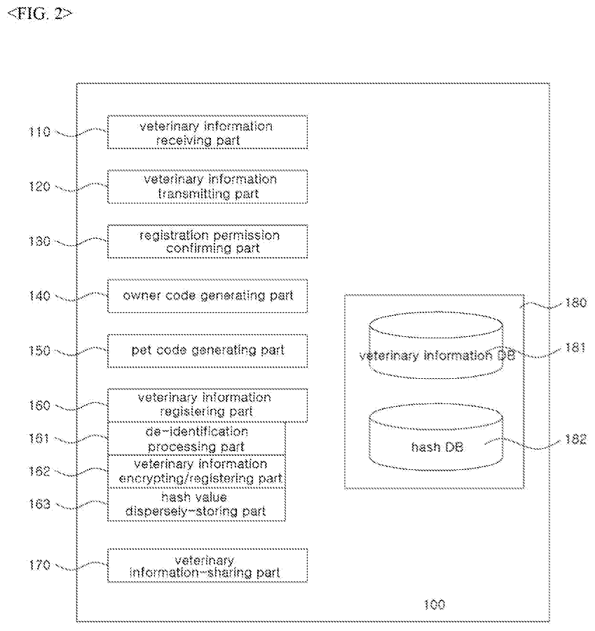

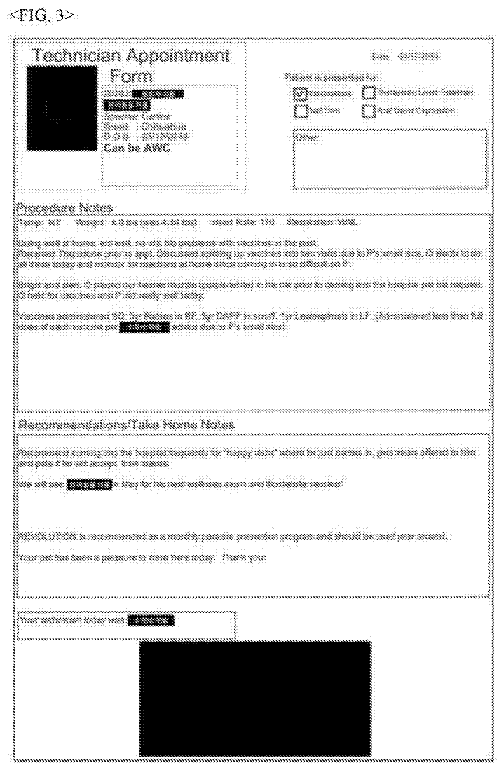

FIG. 1 is a block diagram showing a network configuration of a sharing system of veterinary information for pets according to an embodiment of the present disclosure, FIG. 2 is a block diagram showing a major configuration of the veterinary information sharing system according to an embodiment of the present disclosure, and FIG. 3 is a diagram illustrating an example of veterinary information provided with owner's information processed in a de-identification manner, according to an embodiment of the present disclosure.

First, referring to FIG. 1, a sharing system of veterinary information for pets 100 (hereinafter referred to simply as “veterinary information sharing system 100”) of the present embodiment may be connected to a veterinarian terminal 200 distributed all over the country or distributed in administrative districts, and an owner terminal 300 of an owner who raises a pet, through a communication network, and may also be connected to a study institution terminal 400 of a study institution which requires veterinary information of pets. Each of the veterinarian terminal 200, the owner terminal 300, and the study institution terminal 400 may be provided in plural numbers. The veterinary information sharing system 100, the veterinarian terminal 200, the owner terminal 300, and the study institution terminal 400 constitutes a network connected to each other by a wired or wireless communication network.

The veterinarian terminal 200 may include various terminals, such as a personal computer (PC), a notebook, a tablet computer which is provided in an animal clinic, or a smartphone of a veterinarian, and the owner terminal 300 may include a portable terminal, such as a smartphone of the owner. The study institution terminal 400 may include various types of terminals, such as a server, a personal computer (PC), a notebook, or a tablet computer that is provided in each institution, or a smartphone of a researcher. However, the type of each terminal is not particularly limited.

The veterinary information sharing system 100 according to the present embodiment may be implemented by a collective entity (in the form of an application program) of code data installed and executed in the memory of one computer system or a server computer system. Such an application program may be installed and executed in the veterinarian terminal 200 and the owner terminal 300 in the form of application. In addition, the above-described application program may be installed on the study institution terminal 400 as necessary.

The veterinary information sharing system 100 of the present embodiment basically stores and manages, in the server, veterinary information for pets that is transmitted from the veterinarian terminal 200 located in each animal clinic. At this time, the veterinary information sharing system 100 codes owner's information, and subsequently, matches coded owner's information with veterinary information of pets, and stores and manages matched information.

In addition, the veterinary information sharing system 100 performs a hashing process and an encrypting process on owner's information, pet's information, and detailed veterinary information of the pet, and stores processed information. A hash value obtained by the hashing process is dispersedly stored to enhance the security and integrity of veterinary information.

In addition, in response to a request of a study institution, the veterinary information sharing system 100 provides veterinary information stored in the server to the study institution terminal 400 of the study institution and shares veterinary information with the study institution. At this time, the veterinary information sharing system 100 processes personal information of the owner in a de-identification manner, matches a separately-coded identification code with veterinary information of pets, and provides matched information, thus preventing personal information of the owner from leaking.

As illustrated in FIG. 2, the veterinary information sharing system 100 includes: a veterinary information receiving part 110 configured to receive veterinary information for pets inputted to the veterinarian terminal 200; a veterinary information delivering part 120 configured to deliver the received veterinary information to the owner terminal 300; a registration permission confirming part 130 configured to confirm whether the owner permits the registration and sharing of veterinary information for pets or not; an owner code generating part 140 configured to generate an owner identification code from the received veterinary information; a pet code generating part 150 configured to generate a pet identification code from the received information; a veterinary information registering part 160 configured to register the received veterinary information; a veterinary information sharing part 170 configured to provide veterinary information according to an external request; and a database (DB) server 180 in which various kinds of DBs are stored.

Specifically, the veterinary information receiving part 110 is configured to receive veterinary information inputted through the veterinarian terminal 200 when the pet accompanied by the owner visits a clinic.

When the owner and the pet visit the clinic together, the veterinarian inputs, to the veterinarian terminal 200, basic information including owner's information and pet's information before starting the medical treatment. In the basic information inputted through the veterinarian terminal 200, owner's information may include the name, address, contact details and the like of the owner, and pet's information may include a species of the pet (dog, cat, hamster, rabbit, or the like).

In addition, after the medical treatment, the veterinarian inputs, to the veterinarian terminal 200, detailed veterinary information including biological information of the pet (body information such as breed age, weight, height, blood pressure, muzzle pattern, vein pattern, integument pattern, and the like), disease information of the pet (details about treatment date, causes, symptoms, diagnosis, disease name, prescription, and the like) and the like.

The veterinary information receiving part 110 receives owner's information, pet's information, and the detailed veterinary information of the pet, which are transmitted from the veterinarian terminal 200 as described above and makes a database of the received information. Here, owner's information and pet's information, which are the basic information, may be received through the owner terminal 300 when the application is installed on the owner terminal 300.

The veterinary information delivering part 120 is configured to deliver veterinary information received from the veterinarian terminal 200 to the owner terminal 300. When receiving veterinary information from the veterinarian terminal 200, veterinary information delivering part 120 sends the received veterinary information to the owner terminal 300 so that the owner can confirm veterinary information through the owner terminal 300.

Further, in addition to veterinary information, veterinary information delivering part 120 may further provide registration permission request information for confirming whether the owner permits the registration or sharing of veterinary information or not. The registration permission request information may be provided in the form of an icon or the like. When the owner confirms veterinary information, if the owner wants to register veterinary information in the DB server 180 of the veterinary information sharing system 100, or share veterinary information, the owner clicks the registration permission icon to permit the registration or sharing of veterinary information. Then, the owner terminal 300 sends the permitted registration permission request information to the veterinary information sharing system 100. At this time, the permitted registration permission request information may include request information for permitting only the registration of veterinary information or request information for permitting both the registration of veterinary information and the sharing of veterinary information.

Further, the veterinary information delivering part 120 may be configured to process veterinary information received from the veterinarian terminal 200 in a form that can be easily confirmed by the owner, add an explanation information that describes hard-to-understand medical terminologies, and further provide statistical information about veterinary information of a specific item. To do this, the veterinary information delivering part 120 may include a veterinary information processing part, an explanation information extracting part, and a statistical information calculating part.

In general, veterinary information recorded and provided in a chart form has a large number of insignificant portions that are pointless to the owner. Actual important information is just a few of veterinary information. For this reason, in order to find such important veterinary information through the smartphone having a small screen, the owner needs to directly scroll and find out the respective portion of the actual important information. This may cause inconvenience to the owner. Thus, veterinary information processing part processes veterinary information using, for example, a method of deleting a blank portion and enlarging a character portion, such that the owner can easily confirm veterinary information.

In addition, most of veterinary information recorded by a veterinarian is hard to be understood by the owner, who is a layman in the medical field. Thus, the explanation information extracting part extracts the explanation for information that is described by terms for allowing the layman to understand professional medical terminologies easily. To do this, the DB server may be provided with an explanation DB.

Further, when veterinary information about one or more treatment items is transmitted from the veterinarian terminal 200, the statistical information calculating part extracts the previous veterinary information corresponding to the respective treatment item, compares the extracted previous veterinary information with received veterinary information, converts a trend corresponding to the comparison result into graph information or the like, and provides the result.

Thus, the veterinary information delivering part 120 enables the owner to more easily confirm and understand veterinary information.

The registration permission confirming part 130 is configured to confirm whether permission of information that requests the registration or sharing of veterinary information or not is transmitted from the owner terminal 300. When the registration permission confirming part 130 confirms that the permission for the registration or sharing of veterinary information is transmitted from the owner terminal 300, veterinary information is stored and managed in the DB server 180 and provided to the outside. Since the registration or sharing of veterinary information for pets is performed in accordance with the permission of the owner of the pet, veterinary information for pets can be prevented from being used without permission.

The owner code generating part 140 is configured to generate unique identification codes or identifying a plurality of owners from received owner's information. Thus, different owner codes may be generated for the plurality of owners. As one example, the owner codes may be distinguished by different IDs having regularity, such as ANI001, ANI002, ANI003, and the like, which correspond to the plurality of owners. By identifying the owners with such codes, the name, address, contact details, and the like, which correspond to personal information of the owner, can be processed in a de-identification manner.

The pet code generating part 150 is configured to generate unique identification codes for identifying a plurality of pets from received pet's information. The pet code of the present embodiment may be generated as a species-based serial number of the pet. In one example, the species-based serial number may begenerated as codes such as D01, D02, D03, and the like for dogs, and maybe generated as codes such as C01, C02, C03 and the like for cats.

The owner code and the pet code generated in this manner are matched with each other to form one composite identification code. Specifically, in a case in which an owner having a code ANI001 raises one dog and one cat, a code D01 and a code COI may be assigned to the dog and the cat, respectively. The owner code and the pet code are matched with each other to form composite identification codes such as ANI001-D01 and ANI001-C01. Such composite identification codes, namely the combination of the owner code and the pet code, constitute veterinary information of pets.

In one example, in a case in there are plural pieces of veterinary information for pets having the composite identification code ANI001-D01, veterinary information corresponds to information obtained by subjecting the dog of the code D01 that is being raised by the owner of the code ANI001 to medical treatment multiple times in a clinic. Thus, from the plural pieces of veterinary information of pets, the pet treatment history of the code ANI001-D01 can be utilized as the study material.

In addition, the pet of the code ANI001-D01 is a first dog that is being raised by the owner of the code ANI001, and the pet of the code ANI001-C01 is a first cat that is being raised by the owner of the code ANION. Information representing that the dog and the cat are being raised by the same owner can be provided. Therefore, since information representing that the two pets were raised in the same environment based on veterinary information for pets of the code ANI001-D01 and the code ANI001-C01 is provided, information provided with such composite identification codes can also be utilized as the study material.

The veterinary information registering part 160 is configured to register veterinary information for pets received from the veterinarian terminal 200 in the DB server 190 of the veterinary information sharing system 100. The veterinary information registering part 160 matches the generated owner code or the generated pet code with veterinary information for pets and registers matched information. The veterinary information registering part 160 may sort and register veterinary information into metadata, including owner's information, the owner code and the pet code, and the detailed veterinary information including information such as symptom, diagnosis, prescription, and the like.

In addition, the veterinary information registering part 160 may process and register owner's information in a de-identification manner in order to protect personal information of the owner. If necessary, the veterinary information registering part 160 may process information of the pet or information of the animal clinic in a de-identification manner. To do this, the veterinary information registering part 160 may include a de-identification processing part 161. The de-identification processing part 161 may delete owner's information or perform a blinding process on owner's information such that owner's information is not identified. As one example, when veterinary information of a specific pet is registered, as the metadata, detailed veterinary information is registered together with the metadata including the owner code, the pet code or the composite identification code in which the codes are combined with each other, such as ANI001-D01. In this case, personal information (owner's information), such as the name, address, contact details, and the like of the owner, is processed and registered in a de-identification manner.

Further, in order to enhance security and integrity of veterinary information, the veterinary information registering part 160 may further include a veterinary information encrypting/registering part 162 configured to perform a hashing process and an encrypting process on veterinary information received from the veterinarian terminal 200 and register the same in a veterinary information DB 181, and a hash value dispersedly-storing part 163 configured to match a hash value generated by performing the hashing process on veterinary information with at least one of the owner code, the pet code, and the composite identification code, and dispersedly store the same in a hash DB 182.

When a request to view veterinary information is issued from the study institution terminal 400, the veterinary information sharing part 170 is configured to provide veterinary information to the study institution terminal 400 that issues the view request. The study institution described in the present embodiment may be regarded as a concept that involves institutions and associations which utilize veterinary information for a public or private purpose and may include various kinds of institutions, such as a school, a research institute, a clinic, a raising consulting group, an investigation program and the like.

When a request to view veterinary information is issued, the veterinary information sharing part 170 provides veterinary information for pets included in the request, extracts a hash value that is to be matched with at least one of the owner code, the pet code and the composite identification code, which are included in veterinary information of pets, finds the encrypted veterinary information corresponding to the extracted hash value, decodes the found information and provides the decoded information to the study institution terminal 400.

Veterinary information described in the present embodiment may be classified into the metadata, including information such as owner's information, the owner code and the pet code, and the detailed veterinary information including information such as symptom, diagnosis, prescription, and the like. As illustrated in FIG. 3, the veterinary information sharing part 170, which provides the metadata and the detailed veterinary information, may process personal information, such as the name, address, contact details and the like of the owner, in a de-identification manner, and if necessary, may also process pet's information and information of the animal clinic in a de-identification manner. That is, veterinary information provided from the veterinary information sharing part 170 is identified by the owner code and the pet code. Since owner's information is provided while being processed in a de-identification manner, personal information of the owner is protected.

The DB server 180 stores and manages veterinary information of pets. To do this, the DB server 180 includes the veterinary information DB 181 that stores veterinary information for pets and the hash DB 182 that stores the hash value generated by performing the hashing process on veterinary information in the state in which the hash value is matched with at least one of the owner code, the pet code, and the composite identification code.

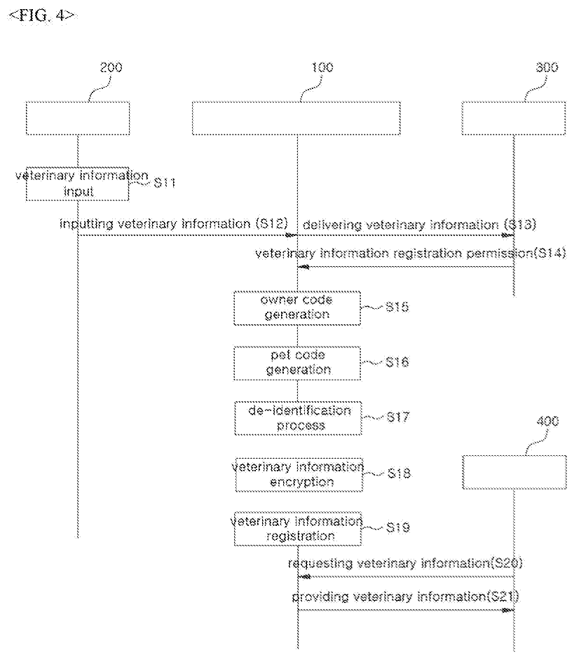

FIG. 4 is a control flowchart illustrating a process of sharing veterinary information for pets according to an embodiment of the present disclosure.

Referring to FIG. 4, when an owner visits an animal clinic together with a pet thereof, a veterinarian gives medical treatment for the pet and inputs respective veterinary information through the veterinarian terminal 200 (S11). The input veterinary information is transmitted to the sharing system of veterinary information for pets 100 (S12). Veterinary information receiving part 110 receives the transmitted veterinary information. The received veterinary information may include owner's information and pet's information in addition to the detailed veterinary information of the pet.

The veterinary information delivering part 120 delivers the received veterinary information to the owner terminal 300 (S13). At this time, the veterinary information delivering part 120 processes veterinary information in a form that can be easily confirmed by the owner, and may further deliver the explanation of information described by easy-to-understand terms and the statistical information of the treatment item. The owner confirms information through the owner terminal 300 and then inputs permission information for permitting the registration or sharing of information into the owner terminal 300. The owner terminal 300 sends the permission information to the veterinary information sharing system 100 (S14).

When receiving the permission information for permitting the registration or sharing of information from the owner terminal 300, the owner code generating part 140 generates an owner code corresponding to owner's information included in the received veterinary information of pets, which is a pre-step of registering veterinary information (S15). The pet code generating part 150 generates a pet code corresponding to pet's information included in the received veterinary information for pets (S16).

Then, the veterinary information registering part 160 encrypts and stores veterinary information for pets together with the generated owner code and the generated pet code in the DB server 180 (S17, 518, and S19). At this time, the owner code and the pet code are combined with each other to form one composite identification code. Information is classified into the metadata, including owner's information, the owner code and the pet code, and the detailed veterinary information including symptom, diagnosis, prescription, and the like. Owner's information is registered while being processed in a de-identification manner.

Further, the veterinary information registering part 160 performs the hashing process and the encrypting process on information and stores processed information in the veterinary information DB 181. The hash value, which is generated by performing the hashing process on veterinary information, is matched with at least one of the owner code, the pet code, and the composite identification code, and dispersedly stored in the hash DB 182.

After veterinary information for pets is stored and registered in the DB server 180 of the veterinary information sharing system 100, when a request is issued from the study institution terminal 400 (S20), the veterinary information sharing part 170 provides information with owner's information processed in a de-identification manner to the study institution terminal 400 (S21). Thus, by providing information excluding personal information of the owner, it is possible to utilize veterinary information for pets while protecting personal information of the owner in a safe manner.

In addition, the veterinary information sharing part 170 extracts the hash value that is to be matched with at least one of the owner code, the pet code and the composite identification code included in veterinary information of pets, finds and decodes encrypted veterinary information corresponding to the extracted hash value, and provides the same to the study institution terminal 400.

While exemplary embodiments of the present disclosure have been illustrated and described as described above, various modifications and other embodiments may be made by those skilled in the art. Such modifications and other embodiments are all contemplated and included in the appended claims without departing from the spirit and scope of the present disclosure.

### EXPLANATION OF REFERENCE NUMERALS

100: veterinary information sharing system
110: veterinary information receiving part
120: veterinary information delivering part
130: registration permission confirming part
140: owner code generating part
150: pet code generating part
160: veterinary information registering part
162: veterinary information encrypting/registering part
163: hash value dispersedly-storing part
170: veterinary information sharing part
180: DB server

What is claimed is:

1. A sharing system of veterinary information for pets for registering a veterinary information for pets transmitted from a veterinarian terminal, and providing the registered veterinary information for pets to a study institution terminal, comprising:
a veterinary information receiving part configured to receive the veterinary information for pets transmitted from the veterinarian terminal;
a veterinary information delivering part configured to deliver the veterinary information for pets received at the veterinary information receiving part to an owner terminal;
an owner code generating part configured to generate an owner code of an owner from owner's information included in the veterinary information of pets;
a pet code generating part configured to generate a pet code from an animal information included in the veterinary information for pets;
a veterinary information registering part configured to register the veterinary information for pets, the owner code, and the pet code in a database server; and
a veterinary information sharing part configured to process, in response to a request from the study institution terminal, owner's information in a de-identification manner, and provide the veterinary information for pets registered in the database server to the study institution terminal.

2. The sharing system according to claim 1, wherein the owner's code and the pet's code are configured to be identified by a composite identification code in which the owner code and the pet code are combined with each other.

3. The sharing system according to claim 2, wherein the pet code is generated using a species-based serial number of the pet.

4. The sharing system according to claim 1, wherein:
the veterinary information delivering part is configured to further transmit, to the owner terminal of the owner, a registration permission request information for confirming whether the owner wants to register or share of the veterinary information for pets or not; and
in response to a permission signal that permits the registration or sharing of the veterinary information for pets, which is transmitted from the owner terminal of the owner, the veterinary information registering part is configured to register the veterinary information for pets, and the veterinary information sharing part is configured to allow the veterinary information for pets to be shared.

5. The sharing system according to claim 1, wherein the veterinary information delivering part includes:
a veterinary information processing part configured to process the veterinary information for pets in a form that is easy to be checked by the owner;
an explanation information extracting part configured to extract explanation information for explaining the veterinary information for pets; and
a statistical information calculating part configured to calculate a statistical information about a treatment item of the veterinary information for pets.

6. The sharing system according to claim 1, wherein the registering part of veterinary information includes:
a de-identification processing part configured to process owner's information included in the veterinary information for pets in a de-identification manner;
a veterinary information encrypting/registering part configured to perform a hashing process and an encrypting process on the veterinary information for pets and register the processed information in a species-based database; and
a hash value dispersedly-storing part configured to match a hash value, which is generated by processing the hashing process on the veterinary information for pets at the veterinary information encrypting/registering part, with at least one of the owner code, the pet code, and the composite identification code, and dispersedly store matched information in a hash database.
